SEQUENCE LISTING (13 sequences)

SEQ ID NO: 1 - DNA, 61 bp, Artificial
  generalized sequence of promoter psaA*
gtcttgactnnnnnnnnnnnnntgntataatnnnnnnnnnnnnnaaggaggcaatcatatg

SEQ ID NO: 2 - DNA, 61 bp, Artificial
  psaA* promoter
gtcttgactagggggggggaggtggtataatctactagtgaattaaggaggcaatcatatg

SEQ ID NO: 3 - DNA, 61 bp, Artificial
  fusion of the -35 sequence and the Pribnow-box of the native psaA promoter with the ribosomal binding site of the isiA promoter
gtcttgcctagggggggggaggccgtattatcttctagtgaattaaggaggcaattctgtg

SEQ ID NO: 4 - DNA, 294 bp, Synechocystis PCC6803
aatttttgtaacaaagttcaactttgtcttgacttttgtaagtctttgcaaaatctaggagctagaactggtcagggctggggcaatttttaattattgttacgcaggtcttgcctagggggggggaggccgtattatcttctagtgatgtttgctgaaaacgcctatctgtgcaaggtttaacatcgttattatgaagcgaaaactaattcccttttttacgcttcctctattacactattctgcataggaaacccttaatagttcattgtcgagcgaggagaaccctgcatg

SEQ ID NO: 5 - DNA, 60 bp, Synechocystis PCC6803
tctcggcacttattgccataatttattatttgtcgtctcaattaaggaggcaattctgtg

SEQ ID NO: 6 - DNA, 61 bp, Artificial
  exchange of four nucleotides from hybrid promoter between PsaA and PisiA
gtcttgactagggggggggaggtggtataatcttctagtgaattaaggaggcaattctgtg

SEQ ID NO: 7 - DNA, 67 bp, Artificial
  introduction of rerstriction sites into PpsaA*
gtcgacgtcttgactagggggggggaggtggtataatctactagtgaattaaggaggcaatcatatg

SEQ ID NO: 8 - DNA, 1695 bp, Artificial
  codon optimized gene of isoprene synthase from Pueraria Montana
atgtgtgctaccagctctcaatttacccaaattaccgaacataatagtcggcgttccgctaattaccagcccaacctctggaattttgaatttctgcaatctctggaaaatgaccttaaggtggaaaaattagaagagaaggccaccaagttagaggaggaggtacggtgcatgatcaaccgggtagacacccaacccttaagcttattagaattgatcgacgatgtccagcgtttaggtttgacctacaagtttgagaaggacattatcaaagcccttgagaatattgttttgctggatgagaataagaaaaataaaagtgacctccatgctactgctctcagcttccgtttacttcggcaacatggctttgaggtttcccaagatgtgtttgagcggtttaaggacaaggagggtggtttcagtggtgaacttaaaggtgatgtgcaagggttgctgagtttatatgaagcctcctatcttggctttgagggtgaaaatctcttggaggaggcccggaccttttccattacccatctcaagaacaacttaaaagaaggtattaacaccaaagtggccgaacaagttagtcatgccctggaacttccctatcatcaacggttgcatcggttagaagcccggtggttccttgacaaatatgaacccaaggaaccccaccatcagttattactggagcttgccaagttagatttcaatatggtgcaaaccttgcaccagaaagaactgcaagacctgtcccggtggtggacggagatggggttagccagcaagttagactttgtccgggatcggttaatggaagtgtatttttgggcgttgggtatggcccctgatcctcaattcggtgaatgtcgtaaagctgtcactaaaatgtttggtttggtcaccatcattgatgatgtatatgacgtttatggtactttggatgagttacaactcttcactgatgctgttgagcggtgggacgtgaatgccattaacacccttcccgactacatgaagttgtgcttcttagccctttataacaccgtcaatgacacgtcttatagcatccttaaagaaaaaggtcacaacaacctttcctatttgaccaaatcttggcgtgagttatgcaaagccttccttcaagaagccaaatggtccaacaacaaaatcattcccgcctttagcaagtacctggaaaatgcctccgtgtcctcctccggtgtggctttgcttgctccttcctacttctccgtgtgccaacaacaagaagatatctccgaccatgctcttcgttctttaactgattttcatggccttgtgcgctcctcctgcgtcattttccggctctgcaatgatttggctacctccgcggctgagttagagcggggtgagacgaccaattccattatttcttatatgcacgagaatgacggcacttctgaagagcaagcccgtgaggagttgcggaaattgattgatgccgagtggaagaagatgaaccgggagcgggtttccgattctaccttactccccaaagcttttatggaaattgctgttaacatggctcgggtttcccattgcacctaccaatatggtgacggtcttggtcggcccgactacgccaccgagaatcggatcaagttgttacttattgacccctttcccatcaatcaattaatgtacgtgtaa

SEQ ID NO: 9 - protein, 564 aa, Pueraria Montana
MCATSSQFTQITEHNSRRSANYQPNLWNFEFLQSLENDLKVEKLEEKATKLEEEVRCMINRVDTQPLSLLELIDDVQRLGLTYKFEKDIIKALENIVLLDENKKNKSDLHATALSFRLLRQHGFEVSQDVFERFKDKEGGFSGELKGDVQGLLSLYEASYLGFEGENLLEEARTFSITHLKNNLKEGINTKVAEQVSHALELPYHQRLHRLEARWFLDKYEPKEPHHQLLLELAKLDFNMVQTLHQKELQDLSRWWTEMGLASKLDFVRDRLMEVYFWALGMAPDPQFGECRKAVTKMFGLVTIIDDVYDVYGTLDELQLFTDAVERWDVNAINTLPDYMKLCFLALYNTVNDTSYSILKEKGHNNLSYLTKSWRELCKAFLQEAKWSNNKIIPAFSKYLENASVSSSGVALLAPSYFSVCQQQEDISDHALRSLTDFHGLVRSSCVIFRLCNDLATSAAELERGETTNSIISYMHENDGTSEEQAREELRKLIDAEWKKMNRERVSDSTLLPKAFMEIAVNMARVSHCTYQYGDGLGRPDYATENRIKLLLIDPFPINQLMYV

SEQ ID NO: 10 - DNA, 11542 bp, Artificial
  plasmid pVZ325-PrbcL-IspSoop
tcgacatcaggaattgtaattagaaagtccaaaaattgtaatttaaaaaacagtcaatggagagcattgccataagtaaaggcatcccctgcgtgataagattaccttcagaaaacagatagttgctgggttatcgcagatttttctcgcaaccaaataactgtaaataataactgtctctggggcgacggtaggctttatattgccaaatttcgcccgtgggagaaagctaggctattcaatgtttatggaggactgaccatatgtgtgctaccagctctcaatttacccaaattaccgaacataatagtcggcgttccgctaattaccagcccaacctctggaattttgaatttctgcaatctctggaaaatgaccttaaggtggaaaaattagaagagaaggccaccaagttagaggaggaggtacggtgcatgatcaaccgggtagacacccaacccttaagcttattagaattgatcgacgatgtccagcgtttaggtttgacctacaagtttgagaaggacattatcaaagcccttgagaatattgttttgctggatgagaataagaaaaataaaagtgacctccatgctactgctctcagcttccgtttacttcggcaacatggctttgaggtttcccaagatgtgtttgagcggtttaaggacaaggagggtggtttcagtggtgaacttaaaggtgatgtgcaagggttgctgagtttatatgaagcctcctatcttggctttgagggtgaaaatctcttggaggaggcccggaccttttccattacccatctcaagaacaacttaaaagaaggtattaacaccaaagtggccgaacaagttagtcatgccctggaacttccctatcatcaacggttgcatcggttagaagcccggtggttccttgacaaatatgaacccaaggaaccccaccatcagttattactggagcttgccaagttagatttcaatatggtgcaaaccttgcaccagaaagaactgcaagacctgtcccggtggtggacggagatggggttagccagcaagttagactttgtccgggatcggttaatggaagtgtatttttgggcgttgggtatggcccctgatcctcaattcggtgaatgtcgtaaagctgtcactaaaatgtttggtttggtcaccatcattgatgatgtatatgacgtttatggtactttggatgagttacaactcttcactgatgctgttgagcggtgggacgtgaatgccattaacacccttcccgactacatgaagttgtgcttcttagccctttataacaccgtcaatgacacgtcttatagcatccttaaagaaaaaggtcacaacaacctttcctatttgaccaaatcttggcgtgagttatgcaaagccttccttcaagaagccaaatggtccaacaacaaaatcattcccgcctttagcaagtacctggaaaatgcctccgtgtcctcctccggtgtggctttgcttgctccttcctacttctccgtgtgccaacaacaagaagatatctccgaccatgctcttcgttctttaactgattttcatggccttgtgcgctcctcctgcgtcattttccggctctgcaatgatttggctacctccgcggctgagttagagcggggtgagacgaccaattccattatttcttatatgcacgagaatgacggcacttctgaagagcaagcccgtgaggagttgcggaaattgattgatgccgagtggaagaagatgaaccgggagcgggtttccgattctaccttactccccaaagcttttatggaaattgctgttaacatggctcgggtttcccattgcacctaccaatatggtgacggtcttggtcggcccgactacgccaccgagaatcggatcaagttgttacttattgacccctttcccatcaatcaattaatgtacgtgtaataacgctcggttgccgccgggcgttttttattcctgcaggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgctgggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagctgtaatccgggcagcgcaacggaacattcatcagtgtaaaaatggaatcaataaagccctgcgcagcgcgcagggtcagcctgaatacgcgtttaatgaccagcacagtcgtgatggcaaggtcagaatagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgagaccgagctcgaattggccgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaattcgagcatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctattctgaccttgccatcacgactgtgctggtcattaaacgcgtattcaggctgaccctgcgcgctgcgcagggctttattgattccatttttacactgatgaatgttccgttgcgctgcccggattacagatcctctagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagctctagag

SEQ ID NO: 11 - DNA, 13418 bp, Artificial
  plasmid pVZ325-Ptac/lacI-IspSoop
tcgaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaattacgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtacatatgtgtgctaccagctctcaatttacccaaattaccgaacataatagtcggcgttccgctaattaccagcccaacctctggaattttgaatttctgcaatctctggaaaatgaccttaaggtggaaaaattagaagagaaggccaccaagttagaggaggaggtacggtgcatgatcaaccgggtagacacccaacccttaagcttattagaattgatcgacgatgtccagcgtttaggtttgacctacaagtttgagaaggacattatcaaagcccttgagaatattgttttgctggatgagaataagaaaaataaaagtgacctccatgctactgctctcagcttccgtttacttcggcaacatggctttgaggtttcccaagatgtgtttgagcggtttaaggacaaggagggtggtttcagtggtgaacttaaaggtgatgtgcaagggttgctgagtttatatgaagcctcctatcttggctttgagggtgaaaatctcttggaggaggcccggaccttttccattacccatctcaagaacaacttaaaagaaggtattaacaccaaagtggccgaacaagttagtcatgccctggaacttccctatcatcaacggttgcatcggttagaagcccggtggttccttgacaaatatgaacccaaggaaccccaccatcagttattactggagcttgccaagttagatttcaatatggtgcaaaccttgcaccagaaagaactgcaagacctgtcccggtggtggacggagatggggttagccagcaagttagactttgtccgggatcggttaatggaagtgtatttttgggcgttgggtatggcccctgatcctcaattcggtgaatgtcgtaaagctgtcactaaaatgtttggtttggtcaccatcattgatgatgtatatgacgtttatggtactttggatgagttacaactcttcactgatgctgttgagcggtgggacgtgaatgccattaacacccttcccgactacatgaagttgtgcttcttagccctttataacaccgtcaatgacacgtcttatagcatccttaaagaaaaaggtcacaacaacctttcctatttgaccaaatcttggcgtgagttatgcaaagccttccttcaagaagccaaatggtccaacaacaaaatcattcccgcctttagcaagtacctggaaaatgcctccgtgtcctcctccggtgtggctttgcttgctccttcctacttctccgtgtgccaacaacaagaagatatctccgaccatgctcttcgttctttaactgattttcatggccttgtgcgctcctcctgcgtcattttccggctctgcaatgatttggctacctccgcggctgagttagagcggggtgagacgaccaattccattatttcttatatgcacgagaatgacggcacttctgaagagcaagcccgtgaggagttgcggaaattgattgatgccgagtggaagaagatgaaccgggagcgggtttccgattctaccttactccccaaagcttttatggaaattgctgttaacatggctcgggtttcccattgcacctaccaatatggtgacggtcttggtcggcccgactacgccaccgagaatcggatcaagttgttacttattgacccctttcccatcaatcaattaatgtacgtgtaataacgctcggttgccgccgggcgttttttattcctgcaggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgctgggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagctgtaatccgggcagcgcaacggaacattcatcagtgtaaaaatggaatcaataaagccctgcgcagcgcgcagggtcagcctgaatacgcgtttaatgaccagcacagtcgtgatggcaaggtcagaatagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgagaccgagctcgaattggccgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaattcgagcatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctattctgaccttgccatcacgactgtgctggtcattaaacgcgtattcaggctgaccctgcgcgctgcgcagggctttattgattccatttttacactgatgaatgttccgttgcgctgcccggattacagatcctctagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagctctagag

SEQ ID NO: 12 - DNA, 11342 bp, Artificial
  plasmid pVZ325-PpsaA*-IspSoop
tcgacgtcttgactagggggggggaggtggtataatctactagtgaattaaggaggcaatcatatgtgtgctaccagctctcaatttacccaaattaccgaacataatagtcggcgttccgctaattaccagcccaacctctggaattttgaatttctgcaatctctggaaaatgaccttaaggtggaaaaattagaagagaaggccaccaagttagaggaggaggtacggtgcatgatcaaccgggtagacacccaacccttaagcttattagaattgatcgacgatgtccagcgtttaggtttgacctacaagtttgagaaggacattatcaaagcccttgagaatattgttttgctggatgagaataagaaaaataaaagtgacctccatgctactgctctcagcttccgtttacttcggcaacatggctttgaggtttcccaagatgtgtttgagcggtttaaggacaaggagggtggtttcagtggtgaacttaaaggtgatgtgcaagggttgctgagtttatatgaagcctcctatcttggctttgagggtgaaaatctcttggaggaggcccggaccttttccattacccatctcaagaacaacttaaaagaaggtattaacaccaaagtggccgaacaagttagtcatgccctggaacttccctatcatcaacggttgcatcggttagaagcccggtggttccttgacaaatatgaacccaaggaaccccaccatcagttattactggagcttgccaagttagatttcaatatggtgcaaaccttgcaccagaaagaactgcaagacctgtcccggtggtggacggagatggggttagccagcaagttagactttgtccgggatcggttaatggaagtgtatttttgggcgttgggtatggcccctgatcctcaattcggtgaatgtcgtaaagctgtcactaaaatgtttggtttggtcaccatcattgatgatgtatatgacgtttatggtactttggatgagttacaactcttcactgatgctgttgagcggtgggacgtgaatgccattaacacccttcccgactacatgaagttgtgcttcttagccctttataacaccgtcaatgacacgtcttatagcatccttaaagaaaaaggtcacaacaacctttcctatttgaccaaatcttggcgtgagttatgcaaagccttccttcaagaagccaaatggtccaacaacaaaatcattcccgcctttagcaagtacctggaaaatgcctccgtgtcctcctccggtgtggctttgcttgctccttcctacttctccgtgtgccaacaacaagaagatatctccgaccatgctcttcgttctttaactgattttcatggccttgtgcgctcctcctgcgtcattttccggctctgcaatgatttggctacctccgcggctgagttagagcggggtgagacgaccaattccattatttcttatatgcacgagaatgacggcacttctgaagagcaagcccgtgaggagttgcggaaattgattgatgccgagtggaagaagatgaaccgggagcgggtttccgattctaccttactccccaaagcttttatggaaattgctgttaacatggctcgggtttcccattgcacctaccaatatggtgacggtcttggtcggcccgactacgccaccgagaatcggatcaagttgttacttattgacccctttcccatcaatcaattaatgtacgtgtaataacgctcggttgccgccgggcgttttttattcctgcaggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgctgggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagctgtaatccgggcagcgcaacggaacattcatcagtgtaaaaatggaatcaataaagccctgcgcagcgcgcagggtcagcctgaatacgcgtttaatgaccagcacagtcgtgatggcaaggtcagaatagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgagaccgagctcgaattggccgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaattcgagcatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctattctgaccttgccatcacgactgtgctggtcattaaacgcgtattcaggctgaccctgcgcgctgcgcagggctttattgattccatttttacactgatgaatgttccgttgcgctgcccggattacagatcctctagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagctctagag

SEQ ID NO: 13 - DNA, 10330 bp, Artificial
  plasmid pVZ325-PpetJ-IspSoop
atagtccaaaagctgactttcccctccatgctctggggggaattgctctggcaactgattaatccactgagcaacagcccaagacacgcaaacaaaaaccaacgtcttggcgatcgccatcggcaccatgaaaccatcgtaaaagctggggaaagaataaaaaacagtggttcaggaattgcattgccatggccacttcacaaacctagccaattttagcttgaccgcaactttgacagattgtcttttgactttgcctggaccgcctcccataataccttcgcgtcttgaagactttatccttgaaaggagaacatatgtgtgctaccagctctcaatttacccaaattaccgaacataatagtcggcgttccgctaattaccagcccaacctctggaattttgaatttctgcaatctctggaaaatgaccttaaggtggaaaaattagaagagaaggccaccaagttagaggaggaggtacggtgcatgatcaaccgggtagacacccaacccttaagcttattagaattgatcgacgatgtccagcgtttaggtttgacctacaagtttgagaaggacattatcaaagcccttgagaatattgttttgctggatgagaataagaaaaataaaagtgacctccatgctactgctctcagcttccgtttacttcggcaacatggctttgaggtttcccaagatgtgtttgagcggtttaaggacaaggagggtggtttcagtggtgaacttaaaggtgatgtgcaagggttgctgagtttatatgaagcctcctatcttggctttgagggtgaaaatctcttggaggaggcccggaccttttccattacccatctcaagaacaacttaaaagaaggtattaacaccaaagtggccgaacaagttagtcatgccctggaacttccctatcatcaacggttgcatcggttagaagcccggtggttccttgacaaatatgaacccaaggaaccccaccatcagttattactggagcttgccaagttagatttcaatatggtgcaaaccttgcaccagaaagaactgcaagacctgtcccggtggtggacggagatggggttagccagcaagttagactttgtccgggatcggttaatggaagtgtatttttgggcgttgggtatggcccctgatcctcaattcggtgaatgtcgtaaagctgtcactaaaatgtttggtttggtcaccatcattgatgatgtatatgacgtttatggtactttggatgagttacaactcttcactgatgctgttgagcggtgggacgtgaatgccattaacacccttcccgactacatgaagttgtgcttcttagccctttataacaccgtcaatgacacgtcttatagcatccttaaagaaaaaggtcacaacaacctttcctatttgaccaaatcttggcgtgagttatgcaaagccttccttcaagaagccaaatggtccaacaacaaaatcattcccgcctttagcaagtacctggaaaatgcctccgtgtcctcctccggtgtggctttgcttgctccttcctacttctccgtgtgccaacaacaagaagatatctccgaccatgctcttcgttctttaactgattttcatggccttgtgcgctcctcctgcgtcattttccggctctgcaatgatttggctacctccgcggctgagttagagcggggtgagacgaccaattccattatttcttatatgcacgagaatgacggcacttctgaagagcaagcccgtgaggagttgcggaaattgattgatgccgagtggaagaagatgaaccgggagcgggtttccgattctaccttactccccaaagcttttatggaaattgctgttaacatggctcgggtttcccattgcacctaccaatatggtgacggtcttggtcggcccgactacgccaccgagaatcggatcaagttgttacttattgacccctttcccatcaatcaattaatgtacgtgtaataacgctcggttgccgccgggcgttttttattcctgcaggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgctgggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagctgtaatccgggcagcgcaacggaacattcatcagtgtaaaaatggaatcaataaagccctgcgcagcgcgcagggtcagcctgaatacgcgtttaatgaccagcacagtcgtgatggcaaggtcagaatagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgagaccgagctcgaattggccgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaattcgagcatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctattctgaccttgccatcacgactgtgctggtcattaaacgcgtattcaggctgaccctgcgcgctgcgcagggctttattgattccatttttacactgatgaatgttccgttgcgctgcccggattacagatcctctagc

(19) United States
(12) Patent Application Publication
(10) Pub. No.: US 2015/0232884 A1
(43) Pub. Date: Aug. 20, 2015

(54) Genetically Enhanced Cyanobacteria for the Production of Isoprene

(71) Applicant: ALGENOL BIOFUELS INC., Fort Myers, FL (US)

(72) Inventor: Ulf DUEHRING, Fredersdorf (DE)

(73) Assignee: ALGENOL BIOFUELS INC., Fort Myers, FL (US)

(21) Appl. No.: 14/640,915

(22) Filed: Mar. 6, 2015

Related U.S. Application Data
(63) Continuation of application No. PCT/EP2012/067534, filed on Sep. 7, 2012.

(51) Int. Cl. C12P 5/00; C12N 1/20; C12N 9/88

(52) U.S. Cl. CPC C12P 5/007; C12N 9/88; C12N 1/20; C12Y 402/03027

(57) ABSTRACT

A cyanobacterium for the production of isoprene having an extrachromosomal plasmid harboring a gene for the production of isoprene. Such a cyanobacterium exhibits a higher isoprene production rate than other conventional strains.

### CROSS-REFERENCE TO RELATED APPLICATIONS

This application is a continuation of International Application No. PCT/EP2012/067534, filed Sep. 7, 2012, which is incorporated herein by reference in its entirety.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

This application contains a sequence listing submitted by EFS-Web, thereby satisfying the requirements of 37 C.F.R. §§1.821-1.825. The sequence listing, created on Sep. 7, 2012, contains thirteen sequences and is 68 KB in size.

### FIELD OF THE INVENTION

This invention is related to the field of production of chemical compounds of interest by using genetically enhanced cyanobacterial cells.

### BACKGROUND OF THE INVENTION

Biofuels, which derive from carbon fixation of carbon dioxide, are gaining increased importance in the fuel area. Similarly, chemical products which have been made from fossil fuels can be made photosynthetically. One of these important products is isoprene which can be produced on an industrial scale by thermal cracking of petroleum naphtha and which is a side product of the production of ethylene. The annual production rate of isoprene amounts to around 800,000 tons. Isoprene is mainly used to produce synthetic rubber (cis-1,4-polyisoprene), adhesives, golf balls, and medical or personal care products such as surgical gloves. There is a need to produce so-called “bio-isoprene” derived from carbon fixation of carbon dioxide within phototrophic organisms. Lindberg et al. reported in 2009 Synechocystis as a model organism for photosynthetic isoprene production (Lindberg P, Park S, Melis A. Engineering a platform for photosynthetic isoprene production in cyanobacteria, Metab Eng. 2010 January; 12(1):70-79). Lindberg et al. replaced the endogenous psbA2 gene of Synechocystis with the gene encoding isoprene synthase via homologous recombination. The observed isoprene production rate was low at about 50 μg per gram dry cell weight per day. Melis et al. describe in the US-patent applications US 2012/135490 and US 2011/0039323 microorganisms containing inter alia chromosomally integrated genes for isoprene synthase. These microorganisms can contain additional genes coding for enzymes producing substrates for the isoprene synthase such as DXS (1-Deoxy-D-xylulose 5-phosphate synthase). The copy number of genes coding for isoprene synthase cannot be controlled in these microorganisms, because these genes are incorporated into a specific single locus in the chromosomes via double homologous recombination. Thus the copy number of the incorporated ispS genes cannot exceed the copy number of the chromosome. The additional genes for isoprene production furthermore greatly complicate the production of these microorganisms.

Therefore there is a need for an improved method for producing isoprene overcoming some of the disadvantages of the above mentioned methods.

It is therefore one object of certain embodiments of the invention to provide genetically enhanced microorganisms for the production of isoprene which are able to produce isoprene in higher amounts and further aspects of the invention are directed to methods for isoprene production using these genetically enhanced microorganisms.

### SUMMARY OF THE INVENTION

The invention described herein discloses a genetically enhanced cyanobacterium for the production of isoprene comprising an extrachromosomal plasmid including a gene for the production of isoprene.

In an embodiment of the invention, a genetically enhanced cyanobacterium capable of producing isoprene is provided, having an extrachromosomal plasmid harboring a gene encoding an enzyme that catalyzes the production of isoprene, where the gene can be under the transcriptional control of a promoter that comprises a ribosomal binding site and a −10 and −35 region from different cyanobacterial promoters. The extrachromosomal plasmid can be, for example, an endogenous plasmid or a heterologous plasmid. The promoter can be, for example, inducible or constitutive. The promoter can be selected from the group consisting of P_{rbc}, P_{tac/lacI}, P_{psaA}, and P_{petJ}. The promoter can comprise a ribosomal binding site from an isiA-promoter and a −10 and −35 region from a psaA promoter. The promoter sequence can comprise SEQ ID NO: 1, where the 3′-ATG can be the start codon of the gene encoding the enzyme that catalyzes the production of isoprene transcriptionally controlled by this promoter.

The promoter can comprise, for example, a nucleic acid sequence having at least 80% sequence identity to SEQ ID NO: 2, where there can be 100% sequence identity at nucleotides 4-9, 26-31, and 45-51 of SEQ ID NO: 2. There can also be a sequence for the termination of transcription located downstream of the gene encoding the enzyme that catalyzes the production of isoprene.

The gene encoding the enzyme that catalyzes the production of isoprene can comprise, for example, codon triplets that have been changed in order to enhance translation in the cyanobacterium compared to the respective wild type gene that encodes the enzyme that catalyzes the production of isoprene. The gene encoding the enzyme that catalyzes the production of isoprene can be, for example, ispS (isoprene synthase—EC 4.2.3.27). The gene encoding the enzyme that catalyzes the production of isoprene can have, for example, at least 80% sequence identity to the nucleic acid sequence of SEQ ID NO: 8. In an aspect of the invention, no further genes involved in isoprene production are present in the cyanobacterium.

The cyanobacterium can be selected, for example, from the group consisting of: Synechocystis, Synechococcus, Anabaena, Chroococcidiopsis, Chlorogloeopsis, Cyanothece, Lyngbya, Phormidium, Nostoc, Spirulina, Arthrospira, Thermosynechococcus BP1, Trichodesmium, Leptolyngbya, Plectonema, Myxosarcina, Pleurocapsa, Oscillatoria, Pseudanabaena, Cyanobacterium, Geitlerinema, Calothrix, Euhalothece, and Scytonema.

In an embodiment of the invention, a genetically enhanced cyanobacterium capable of producing isoprene is provided, having an extrachromosomal plasmid having a gene encoding an enzyme that catalyzes the production of isoprene where the gene can be under the transcriptional control of a promoter having a sequence of AAGGAGG at its ribosomal binding site, TTGACT at its −10 region, and TATAAT at its −35 region.

In another embodiment of the invention, a genetically enhanced cyanobacterium capable of producing isoprene is provided, having an endogenous extrachromosomal plasmid having a gene encoding an enzyme that catalyzes the production of isoprene.

In yet another embodiment of the invention, a method for producing isoprene is be provided, by culturing the isoprene-producing cyanobacterium described herein in a culture medium, and then separating the isoprene from the genetically enhanced cyanobacterium and the culture medium.

### BRIEF DESCRIPTION OF THE DRAWINGS

In the following further embodiments of the invention will be explained in more detail with reference to the figures and experimental data.

FIG. 1A through FIG. 1B (SEQ ID NO: 8) depicts a codon optimized version of the wild type ispS gene from Pueraria montana. For ease in viewing, the DNA sequence is divided into a first portion (shown in FIG. 1A) and a second portion (shown in FIG. 1B).

FIG. 2 shows a plasmid map of the plasmid pVZ325-P_{rbcL}-IspSₒₒₚ. The nucleotide sequence of this plasmid is SEQ ID NO: 10. This plasmid contains from nucleotides 8396 to 8929 the Gentamycin resistance cassette, from nucleotides 6714 to 6995, the gene mobC, from nucleotides 4389 to 6515 the gene mobA, from nucleotides 5357 to 5767 the gene mob, from nucleotides 4386 to 5357 the gene repB, from nucleotides 4113 to 4325 protein\E, from nucleotides 3905 to 4111 the repressor\protein\F, from nucleotides 3039 to 3875 the gene repA, from nucleotides 2198 to 3049 the gene repC, from nucleotides 264 to 1958 the ispS gene from Pueraria montana (kudzu vine) codon optimized for Synechocystis PCC 6803, from nucleotides 1959 to 1990 the oop terminator, from nucleotides 5 to 262 the native rbcL promoter from Synechocystis PCC 6803, and from nucleotides 10473 to 11481 the Spectinomycin/Streptomycin resistance cassette.

FIG. 3 depicts a plasmid map of the plasmid pVZ325-P_{tac/lacI}-IspSₒₒₚ. The nucleotide sequence of this plasmid is SEQ ID NO: 11. This plasmid contains from nucleotides 4074 to 4925 the gene repC, from nucleotides 4915 to 5751 the gene repA, from nucleotides 5781 to 5987 the repressor\protein\F, from nucleotides 5989 to 6201 protein\E, from nucleotides 6262 to 7233 the gene repB, from nucleotides 7233 to 7643 the gene mob, from nucleotides 6265 to 8391 the gene mobA, from nucleotides 8590 to 8871 the gene mobC, from nucleotides 10272 to 10805 the Gentamycin resistance cassette, from nucleotides 2140 to 3834 the ispS gene from Pueraria montana (kudzu vine) codon optimized for Synechocystis PCC 6803, from nucleotides 3835 to 3866 the oop-terminator, from nucleotides 12349 to 13357 the Spectinomycin/Streptomycin resistance cassette, from nucleotides 231 to 1313 the lac repressor, and from nucleotides 2065 to 2138 the promoter P_{tac} from E. coli.

FIG. 4 is the plasmid map of the vector pVZ325-P_{psaA*}-IspSₒₒₚ. The nucleotide sequence of this plasmid is SEQ ID NO: 12. This plasmid contains from nucleotides 8196 to 8729 the Gentamycin resistance cassette, from nucleotides 6514 to 6795 the gene mobC, from nucleotides 4189 to 6315 the gene mobA, from nucleotides 5157 to 5567 the gene mob, from nucleotides 4186 to 5157 the gene repB, from nucleotides 3913 to 4125 protein\E, from nucleotides 3705 to 3911 the repressor\protein\F, from nucleotides 2839 to 3675 the gene repA, from nucleotides 1998 to 2849 the gene repC, from nucleotides 64 to 1758 the ispS gene from Pueraria montana (kudzu vine) codon optimized for Synechocystis PCC 6803, from nucleotides 6 to 60 P_{psaA*}, the artificial promoter based on psaA promoter and RBS from isiA gene, from nucleotides 1759 to 1790 the oop-terminator, and from nucleotides 10273 to 11281 the Spectinomycin/Streptomycin resistance cassette.

FIG. 5 shows the plasmid map of the vector pVZ325-P_{petJ}-IspSₒₒₚ. The nucleotide sequence of this plasmid is SEQ ID NO: 13. This plasmid contains from nucleotides 2252 to 3103 the gene repC, from nucleotides 3093 to 3929 the gene repA, from nucleotides 3959 to 4165 the repressor\protein\F, from nucleotides 4167 to 4379 protein\E, from nucleotides 4440 to 5411 the gene repB, from nucleotides 5411 to 5821n the gene mob, from nucleotides 4443 to 6569 the gene mobA, from nucleotides 6768 to 7049 the gene mobC, from nucleotides 1 to 316 petJ, the native promoter of petJ gene from Synechocystis PCC 6803, from nucleotides 8450 to 8983 the Gentamycin resistance cassette, from nucleotides 2013 to 2044 the oop-terminator, and from nucleotides 318 to 2012 the ispS gene from Pueraria montana (kudzu vine) codon optimized for Synechocystis PCC 6803.

FIG. 6 shows the calibration curve for determining the amount of isoprene via the GC measurements.

FIG. 7 shows the methylerythritol 4-phosphate pathway finally leading to isoprene via conversion of dimethylallyl pyrophosphate (DMAPP) to isoprene by isoprene synthase.

### DESCRIPTION OF THE INVENTION

Higher isoprene production can be achieved by providing genetically enhanced cyanobacteria for the production of isoprene comprising an extrachromosomal plasmid including a gene for the production of isoprene.

Surprisingly, it was found that the integration of the gene for the production of isoprene into an extrachromosomal plasmid, which is located outside the chromosomes, can result in much higher isoprene production rates compared to the prior art. In particular, up to sixty-fold higher isoprene production rates can be achieved by including the gene for isoprene production on an extrachromosomal plasmid in comparison to the prior art document Lindberg et al. At the same time isoprene production was found to be stable for at least 26 hours.

Extrachromosomal plasmids are circular DNA molecules that are separate from the chromosomes, and which are able to replicate independently of the chromosomal DNA of the cyanobacterium. The typical plasmid sizes vary from 1 to over 1000 kilobase pairs and the number of identical plasmids in a single cell can range from just one copy per cell to a couple of thousand copies per cell.

According to another embodiment of the invention, the extrachromosomal plasmid can be a heterologous plasmid, which is not derived from the cyanobacterial host cell.

One example of a heterologous extrachromosomal plasmid is the pVZ plasmid which can ensure that a high production rate for isoprene can be achieved in the case that the gene for isoprene production is included on such a heterologous plasmid.

According to another embodiment of the invention, the gene for the production of isoprene can also be integrated into an endogenous plasmid of the cyanobacterial host cell. For example it is known that the cyanobacterium Synechococcus PCC 7002 contains six endogenous plasmids having different numbers of copy in the cyanobacterial cell (Xu et al.: “Expression of genes in cyanobacteria: Adaption of Endogenous Plasmids as platforms for High-Level gene Expression in Synechococcus PCC 7002”, Photosynthesis Research Protocols, Methods in Molecular Biology, 684, pages 273 to 293 (2011)). The endogenous plasmid pAQ1 is present in a number of 50 copies per cell (high-copy), the plasmid pAQ3 with 27 copies, the plasmid pAQ4 with 15 copies and the plasmid pAQ5 with 10 copies per cell (low-copy) whereas the chromosome has 6 copies per cell. The great advantage of incorporating the production gene for isoprene production into endogenous extrachromosomal plasmids of the cyanobacterium is that by the choice of the endogenous plasmid used for integration, the number of copies of these genes in the cyanobacterium can easily be controlled, depending on the copy number of the specific endogenous plasmid that is used for that purpose in the cyanobacterium. For example, a higher number of copies of the gene for isoprene production can be achieved via integration into the plasmid pAQ3 in comparison to integration into the plasmid pAQ4 with a lower number of copies in the cell. Additionally, there can be a higher transcription efficiency if the gene for the production of isoprene is encoded on a plasmid in comparison to the chromosome (due to position effects or different condensation levels of the extrachromosomal DNA versus the chromosomal DNA). This could lead to higher expression levels if encoded on the plasmid in comparison to the chromosome even if the copy number and gene dosage, respectively, are the same.

The gene for production of isoprene can be either under the transcriptional control of an inducible or constitutive promoter. One example for a constitutive promoter is the promoter P_{rbc} which is a cyanobacterial promoter controlling the transcription of the ribulose-1,5-bisphosphate carboxylase/oxygenase (Rubisco) enzyme from various cyanobacteria such Synechocystis sp. PCC 6803 (rbcL gene in Synechocystis sp. PCC 6803: slr0009).

Further examples of inducible promoters are the promoters P_{psaA} and P_{petJ}. The promoter P_{psaA} is a light-dependent promoter, for example from the cyanobacterium Synechocystis sp. PCC 6803 which in the wild-type cyanobacterium controls the transcription of the psaA gene: slr1834 (encoding P700 apoprotein subunit Ia). The promoter P_{petJ} is induced under copper starvation conditions and in the wild-type cyanobacterial strain of Synechocystis sp. PCC 6803 controls the transcription of the P_{petJ} gene encoding cytochrome c553 (petJ gene in Synechocystis sp. PCC 6803: sll1796). Another example of an inducible promoter is the prominent P_{tac/lacI} promoter which is often used for heterologous expression in E. coli and can be induced by the addition of isopropyl thiogalactoside.

Prokaryotic promoter sequences in general contain promoter elements with different functions, the −10 promoter element, also called the Pribnow box, which is essential to start transcription and further the −35 element which usually consists of nucleotides of the sequence TTGACA or TTGACT which allows for a very high transcription rate. Downstream of these elements a ribosomal binding site can be found which will serve as a starting point for translation from the messenger RNA.

A higher production rate for isoprene can be achieved if the promoter driving the transcription of the gene for isoprene production is a recombinant promoter comprising promoter elements from different cyanobacterial promoters. In particular, the recombinant promoter can contain a ribosomal binding site from one cyanobacterial promoter and a −10 and a −35 region from a different cyanobacterial promoter. For example, the −10 and −35 region from the light-inducible P_{psaA} promoter can be used in addition with a ribosome binding site from the promoter P_{isiA} (sll0247, iron-stress chlorophyll-binding protein, homologous to psbC). In addition specific nucleotide exchanges can be introduced into or adjacent to the −10 and −35 regions respectively. Very high isoprene production rates can be achieved by using these kind of promoters in the extrachromosomal plasmids. For example, the recombinant promoter can comprise the following generalized nucleic acid sequence (SEQ ID NO: 1):

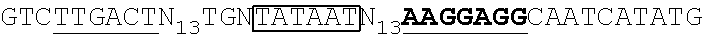

wherein the underlined sequence is the −35 sequence, the boxed sequence is the Pribnow-box and the boldfaced underlined sequence is the ribosomal binding site and each of the nucleotides N is independently selected from A, T, C and G and wherein the 3′-ATG is the start codon of the gene for the production of isoprene transcriptionally controlled by this promoter.

Furthermore, the recombinant promoter can comprise a promoter having at least 80%, or 85% preferably at least 90% or 95%, sequence homology or is identical to the following nucleic acid sequence called P_{psaA*} (SEQ ID NO: 2):

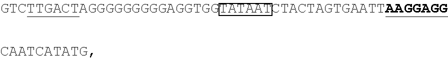

with the proviso that the −35 sequence, the Pribnow-box including the TGG directly upstream adjacent to the Pribnow-box, which has influence on the strength of the P_{psaA*} promoter and the ribosomal binding site of the homologous promoter are identical to the above nucleic acid sequence.

The above described recombinant promoter sequence P_{psaA*} can be generated from the native promoter sequence psaA and the ribosomal binding site of P_{isiA} by the following steps:

step 1) fusion of the −35 sequence and the Pribnow-box of the native psaA promoter via DNA synthesis with the ribosomal binding site of the isiA promoter, thereby generating a chimeric P_{psaA/RBSisiA} (SEQ ID NO: 3):

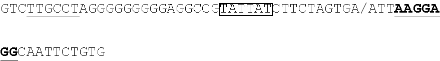

wherein the underlined sequence is the −35 sequence, the boxed sequence is the Pribnow-box both of P_{psaA} and the boldfaced underlined sequence is the ribosomal binding site of P_{isiA}.

The native psaA promoter is denoted by the following sequence (SEQ ID NO: 4), wherein the underlined sequence is the −35 sequence, the boxed sequence is the Pribnow-box, the boldfaced underlined sequence is the ribosomal binding site and the 3′-ATG is the start codon of the gene transcriptionally controlled by the psaA promoter:

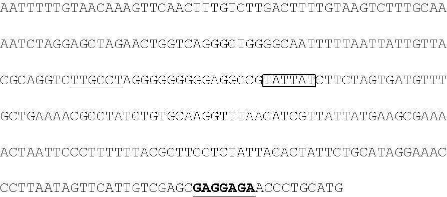

The nucleotide sequence of the native ribosomal binding site of P_{isiA} is as follows (SEQ ID NO: 5):

TCTCGGCACTTATTGCCATAATTTATTATTTGTCGTCTCAATT
AAGGAGGCAATTCTGTG

wherein the ribosomal binding site is marked as the boldfaced underlined sequence and the 3′-GTG is the start codon of the isiA gene.

In a second step four specific boldfaced nucleotides are exchanged in the chimeric sequence of step 1) in order to enhance the promoter strength (SEQ ID NO: 6):

Step 2)

GTCTTGACTAGGGGGGGGGAGGTGGTATAATCTTCTAGTGAAT
TAAGGAGGCAATTCTGTG

In a last step 3) specific restriction sites for cloning are introduced, in particular a SalI (boxed), an SpeI (boldfaced) and an NdeI (underlined) restriction site (SEQ ID NO: 7):

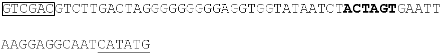

The above described recombinant promoters can ensure very high isoprene production rates of at least 100, preferably 200 or 300, most preferred at least 360 μg isoprene per gDW and hour (gDW—gram dried cell weight) or given in volumetric units of at least 20, preferably 40 or 80, most preferred at least 120 μg isoprene per liter of culture and hour.

In order to further allow for an efficient termination of the transcription and in order to enhance transcript stability downstream of the gene for isoprene production, a terminator sequence can be present for termination of transcription. For example, this terminator sequence can be the oop-terminator from the lambda phage.

In order to enhance the translation efficiency, codon optimized versions of the gene for isoprene production can be inserted into the extrachromosomal plasmid. These nucleotide changes can either be conservative nucleotide changes which only affect the third “wobble base” of the triplet codon coding for the amino acids, which does not change the sequence of the protein for isoprene production encoded by the gene, or can be changes in the nucleotide sequence which can be lead to mutations in the amino acid sequence involving non-essential amino acids, which do not change the enzymatic activity and affinity of the protein for isoprene production encoded by this gene.

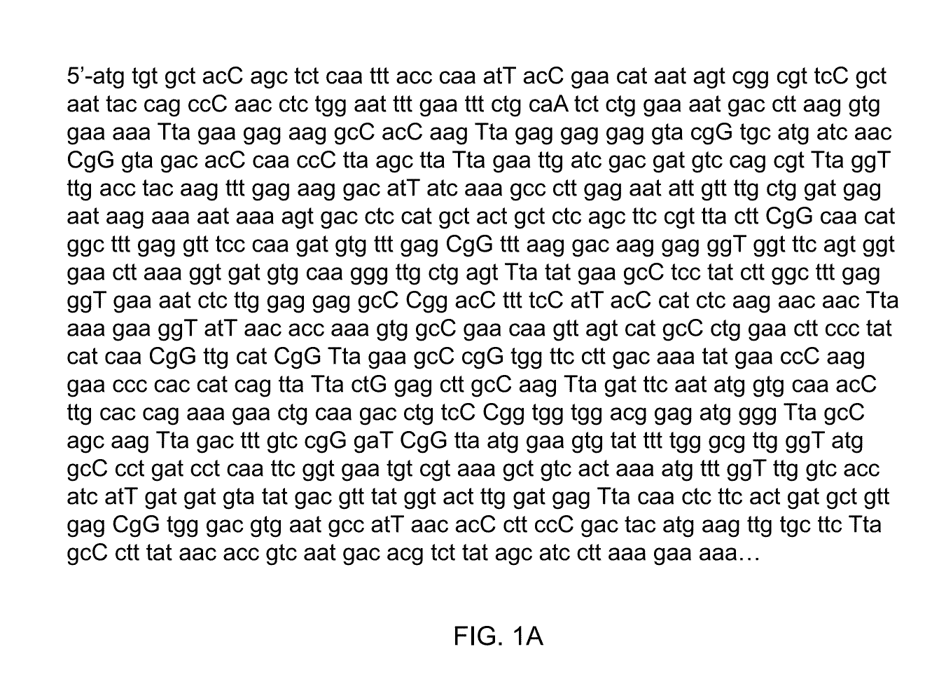

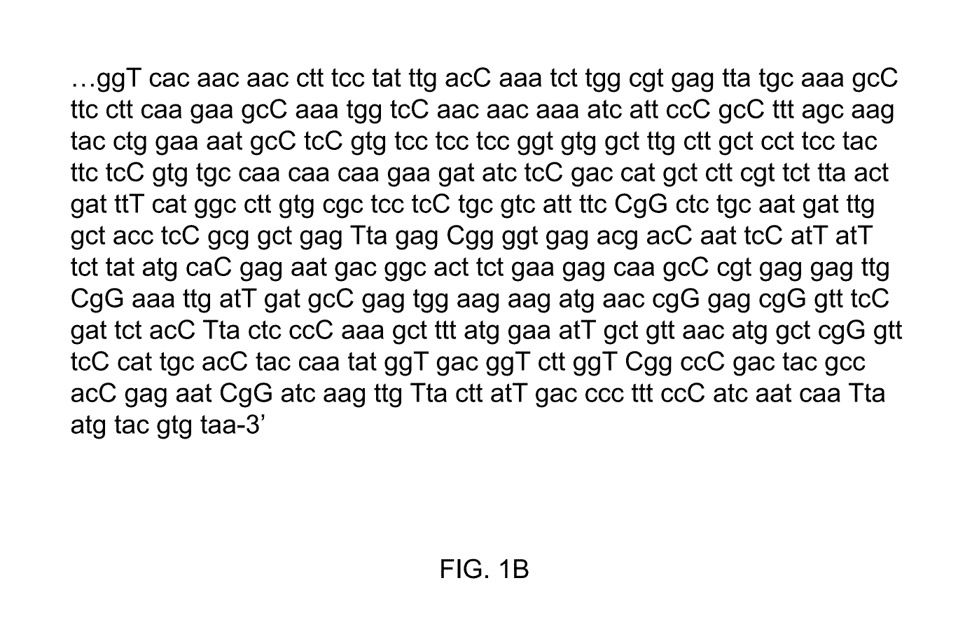

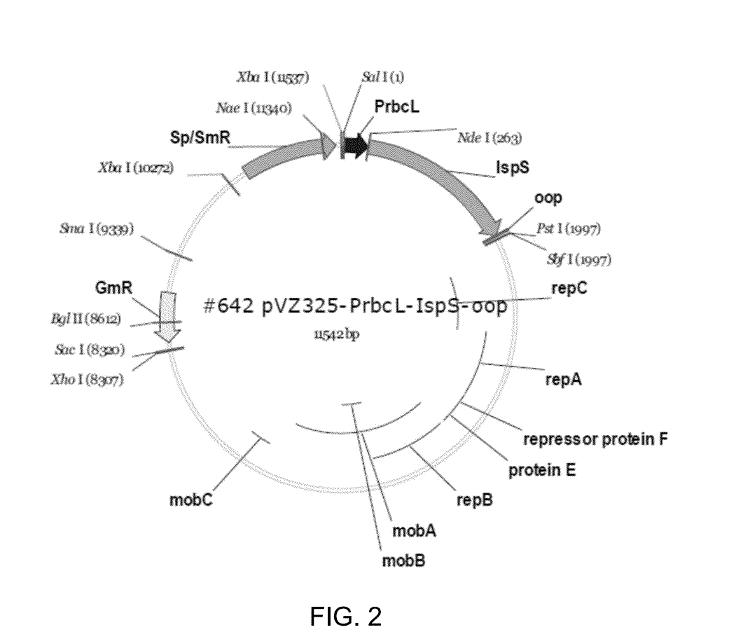

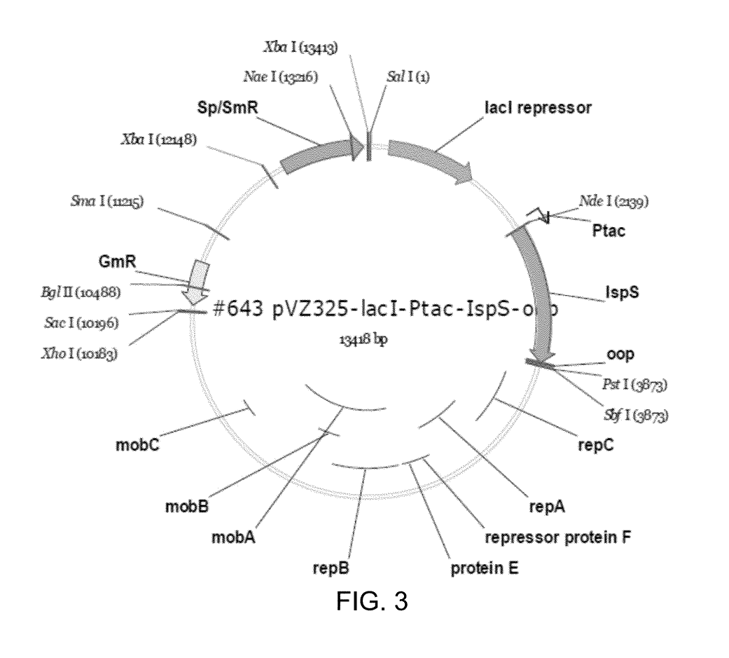

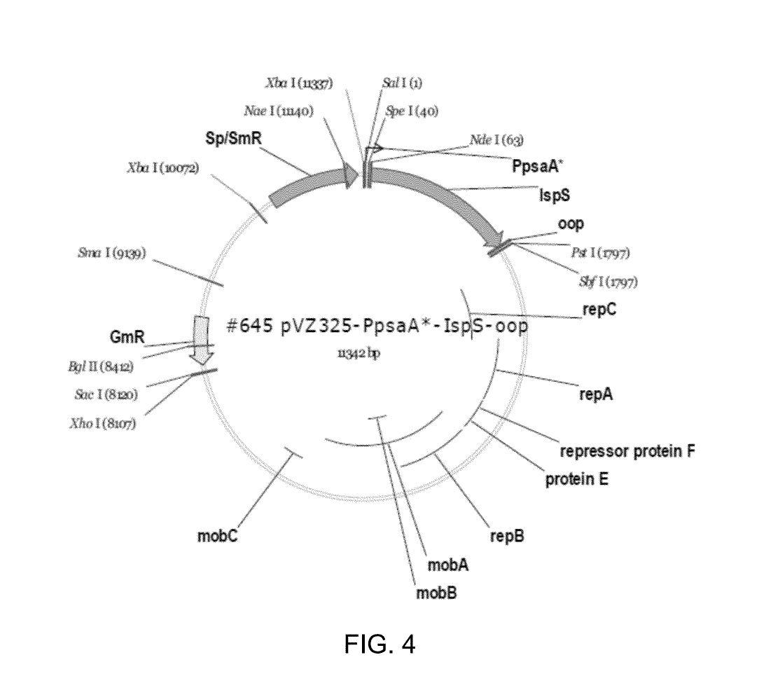

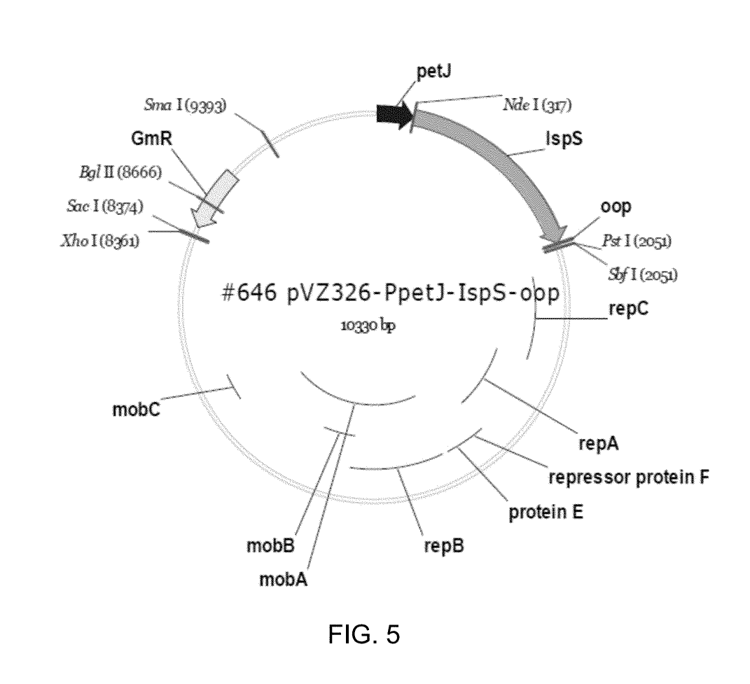

One preferred gene for the production of isoprene is the ispS gene coding for isoprene synthase, an enzyme which converts dimethylallyl phosphate derived from the methylerythritol pathway to isoprene. The gene for the production of isoprene can have at least 80% or 85%, preferably at least 90% or 95% sequence homology or is identical to the following nucleic acid sequence shown in FIG. 1 (SEQ ID NO: 8). In this nucleotide sequence the capitalized letters denote nucleotide changes in comparison to the wild type ispS gene from Pueraria montana (Gen Bank accession no: AY316691). This nucleotide sequence codes for the IspS protein of the SEQ ID NO: 9.

Apart from the gene for the production of isoprene, which codes for an enzyme directly producing isoprene, such as isoprene synthase, no further recombinant genes involved in isoprene production need to be present in the genetically enhanced cyanobacterium according to one embodiment of the invention. In particular, the genetically enhanced cyanobacterium can lack additional recombinant genes of the methylerythritol pathway such as genes coding for enzymes producing substrates for isoprene synthase for example DXS.

Therefore according to this embodiment of the invention, the introduction of one gene for isoprene production is sufficient in order to achieve a high isoprene production rate, which greatly simplifies the production of these genetically enhanced cyanobacteria.

The genetically enhanced cyanobacterium can be selected from a group of various cyanobacteria consisting of: Synechocystis, Synechococcus, Anabaena, Chroococcidiopsis, Chlorogloeopsis, Cyanothece, Lyngbya, Phormidium, Nostoc, Spirulina, Arthrospira, Thermosynechococcus BP1, Trichodesmium, Leptolyngbya, Plectonema, Myxosarcina, Pleurocapsa, Oscillatoria, Pseudanabaena, Cyanobacterium, Geitlerinema, Calothrix, Euhalothece, and Scytonema.

Another variant of the invention is directed to a method for producing isoprene, comprising the following methods steps:
A) culturing the genetically enhanced cyanobacteria as described above in a culture medium, the cyanobacteria thereby producing isoprene,
B) separating the isoprene from the genetically enhanced cyanobacterium and the culture medium.

In particular, the culture medium can be BG11 medium. Depending on whether in the method step A) genetically enhanced cyanobacteria with an extrachromosomal plasmid harboring the gene for isoprene production under an inducible or constitutive promoter are used, method step A) also may comprise a further substep A1) of inducing the isoprene production by adding a certain stimulant to the culture medium leading to an induction of isoprene production. Upon induction in the case of an inducible promoter or even upon culturing in the culture medium in the case of a constitutive promoter, the genetically enhanced cyanobacteria of the invention will produce isoprene which is excreted into the culture medium and in particular into the gas space above the culture medium and which therefore can be detected in the gas space.

In order to allow for efficient photosynthesis of the genetically enhanced cyanobacteria, they can be subjected to light, in particular sunlight and CO₂ so that isoprene can easily be produced via photosynthesis.

### DEFINITIONS AND GENERAL EXPLANATIONS

Database entry numbers given in the following are for the CyanoBase, the genome database for cyanobacteria (http://bacteria.kazusa.or.jp/cyanobase/index.html); Yazukazu et al. “CyanoBase, the genome database for Synechocystis sp. PCC 6803: status for the year 2000”, Nucleic Acid Research, 2000, Vol. 18, page 72.

The EC numbers cited throughout this patent application are enzyme commission numbers which is a numerical classification scheme for enzymes based on the chemical reactions which are catalyzed by the enzymes.

As used herein, the term “genetically enhanced” refers to any change in the endogenous genome of a wild type cyanobacterial cell or to the addition of endogenous and non-endogenous, exogenous genetic code to a wild type cyanobacterial cell, for example the introduction of a heterologous gene. More specifically, such changes are made by the hand of man through the use of recombinant DNA technology or mutagenesis. The changes can involve protein coding sequences or non-protein coding sequences in the genome such regulatory sequences as non-coding RNA, antisense RNA, promoters or enhancers. Aspects of the invention utilize techniques and methods common to the fields of molecular biology, microbiology and cell culture. Useful laboratory references for these types of methodologies are readily available to those skilled in the art. See, for example, Molecular Cloning: A Laboratory Manual (Third Edition), Sambrook, J., et al. (2001) Cold Spring Harbor Laboratory Press; Current Protocols in Microbiology (2007) Edited by Coico, R, et al., John Wiley and Sons, Inc.; The Molecular Biology of Cyanobacteria (1994) Donald Bryant (Ed.), Springer Netherlands; Handbook Of Microalgal Culture: Biotechnology And Applied Phycology (2003) Richmond, A.; (ed.), Blackwell Publishing; and “The cyanobacteria, molecular Biology, Genomics and Evolution”, Edited by Antonia Herrero and Enrique Flores, Caister Academic Press, Norfolk, UK, 2008.

It is well known to a person of ordinary skill in the art that large plasmids can be produced using techniques such as the ones described in US patents U.S. Pat. No. 6,472,184 B1, titled “method for producing nucleic acid polymers,” and U.S. Pat. No. 5,750,380, titled “DNA polymerase mediated synthesis of double stranded nucleic acid molecules,” which are hereby incorporated in their entirety.

Denominations of genes are presented in a three letter lower case name followed by a capitalized letter if more than one related gene exists, for example ispS. The respective protein encoded by that gene is denominated by the same name with the first letter capitalized, such as IspS.

Denominations for promoter sequences, which control the transcription of a certain gene in their natural environment are given by a capitalized letter “P” followed by the gene name according to the above described nomenclature, for example “PpetJ” for the promoter controlling the transcription of the petJ gene.

The term “nucleic acid” is intended to include nucleic acid molecules, such as polynucleotides which include an open reading frame encoding a polypeptide, and can further include non-coding regulatory sequences of genes, such as promoters and enhancers as well as non-coding RNAs. In addition, the terms are intended to include one or more genes that are part of a functional operon. In addition the terms are intended to include a specific gene for a selected purpose. The gene can be endogenous to the host cell or can be recombinantly introduced into the host cell.

In a further aspect, the invention also provides nucleic acids which are at least 60%, 70%, 80%, 90% or 95% identical to the promoter nucleic acids or to the nucleic acids encoding either the proteins for the first essential or conditionally essential genes or the first production genes for the production of the first chemical compound disclosed therein. With regard to the promoters, truncated versions of the promoters including only a small portion of the native promoters upstream of the transcription start point, such as the region ranging from −35 to the transcription start can often be used. The invention also provides amino acid sequences for enzymes for the production of first chemical compounds, which are at least 60%, 70%, 80%, 90% or 95% identical to the amino acid sequences disclosed therein.

The percentage of identity of two nucleic acid sequences or two amino acid sequences can be determined using the algorithm of Thompson et al. (Clustal W, 1994 Nucleic Acid Research 22: pages 4,673 to 4,680). A nucleotide sequence or an amino acid sequence can also be used as a so-called “query sequence” to perform a nucleic acid or amino acid sequence search against public nucleic acid or protein sequence databases in order to, for example identify further unknown homologous promoters, or homologous protein sequences and nucleic acid sequences which can also be used in embodiments of this invention. In addition, any nucleic acid sequences or protein sequences disclosed in this patent application can also be used as a “query sequence” in order to identify yet unknown sequences in public databases, which can encode for example new enzymes which could be useful in this invention. Such searches can be performed using the algorithm of Karlin and Altschul (1999 Proceedings of the National Academy of Sciences USA 87: pages 2264 to 2268), modified as in Karlin and Altschul (1993 Proceedings of the National Academy of Sciences USA, 90: pages 5873 to 5877). Such an algorithm is incorporated in the Nblast and Xblast programs of Altschul et al. (1999 Journal of Molecular Biology 215, pages 403 to 410) Suitable parameters for these database searches with these programs are, for example, a score of 100 and a word length of 12 for blast nucleotide searches as performed with the Nblast program. Blast protein searches are performed with the Xblast program with a score of 50 and a word length of 3. Where gaps exist between two sequences, gapped blast is utilized as described in Altschul et al. (1997 Nucleic Acid Research, 25: pages 3389 to 3402).

The term “genome” refers to the genome of the cyanobacterium including the chromosomal genome as well as the endogenous extrachromosomal plasmids which are normally present in the wild type cyanobacterium. For example, cyanobacteria such as Synechococcus PCC7002 can include up to 6 extrachromosomal plasmids in their wild type form.

### DETAILED DESCRIPTION OF EMBODIMENTS

Cultivation experiments were performed with the following four different isoprene producing strains of Synechocystis sp. PCC 6803 containing the following extrachromosomal plasmids:
1. pVZ325-P_{rbcL}-IspSₒₒₚ (supplemented with 5 μg/ml gentamycin)
2. pVZ325-P_{tac/lacI}-IspSₒₒₚ (supplemented with 5 μg/ml gentamycin & 1 mM IPTG)
3. pVZ325-P_{psaA*}-IspSₒₒₚ (supplemented with 5 μg/ml gentamycin)
4. pVZ325-P_{petJ}-IspSₒₒₚ (supplemented with 5 μg/ml gentamycin, without copper)

### EXAMPLES

### Example 1

### Construct Design

The ispS gene from Pueraria montana (kudzu vine) was taken for DNA synthesis (by GeneArt, Regensburg, Germany). Sequence information was taken from the NCBI nucleotide data base. According to Lindberg et al. (2009) the coding sequence for the predicted chloroplast transit peptide was removed. To assure a high expression level of a foreign gene in Synechocystis rarely used codons within the ispS coding sequence were changed to more frequently used codons. The core element of the psaA promoter was fused in front of the start codon of the synthetic ispS coding sequence, containing the −10 and −35 region and the transcriptional start point as well as the introduced ribosome binding site from the isiA-promoter. Behind the stop codon of the ispS gene the oop terminator (phage lambda) was fused for efficient termination of transcription and increased transcript stability.

This construct was cloned into a vector resulting in the vector pVZ325-P_{psaA*}-IspSₒₒₚ shown in FIG. 1 wherein the ispS gene is under the transcriptional control of the recombinant promoter P_{psaA*} including the ribosomal binding site from P_{isiA} and the −10 and −35 region from P_{psaA}.

The further vectors pVZ325-P_{petJ}-IspSₒₒₚ, pVZ325-P_{tac/lacI}-IspSₒₒₚ, and pVZ325-P_{rbcL}-IspSₒₒₚ were constructed by cloning the respective promoters with SalI/NdeI restriction sites into the vector pVZ325-P_{psaA*}-IspSₒₒₚ thereby the promoter P_{psaA*} was removed.

### Example 2

### Cultivation Conditions

Strains of Synechocystis sp. PCC 6803 were cultivated either on BG11 plates or in Erlenmeyer flasks filled with 50 ml BG11 medium supplemented with 10 μg/ml gentamycin at 28° C. The glass vessels used for the isoprene production assay were gas tight and had a diameter of about 1.5 cm and a total capacity of 20 ml. The vessel was filled with 2 ml culture suspension (BG11) with an optical density between 1-2 at 750 nm which corresponds to a dried cell weight (DW) of 0.14-0.28 g/L. The headspace volume was enriched with CO₂ to an end concentration of 11% (v/v). The light intensity was set to about 100 μE and cells were constantly mixed by stirring with a magnetic stir bar (length: 1 cm). At several time points gas samples of 500 μl were taken from the headspace of the culture vessel and analyzed by gas chromatography.

### Example 3

### Calibration of the Gas Chromatograph and Quantification of Isoprene from the Gas Phase

120 μl of chilled (4° C.) liquid isoprene (density 0.681 g/cm³) were injected with a pre-cooled syringe into a gas sampling tube (1200 ml) via a septum. 2 ml of glass beads (1 mm diameter) were placed inside the gas sampling tube to enable fast and even distribution of the isoprene air mixture before further dilution. After vigorous extended shaking, 5.9 ml of the isoprene air mixture was transferred with a syringe to a 590 ml gas sampling tube with 1 ml glass beads. After vigorous extended mixing, another dilution was performed by transferring another 5.9 ml from the second gas sampling tube to a new gas sampling tube of 590 ml total volume with 1 ml glass beads. Table 1 summarizes the various dilutions:

TABLE 1

| | | Total volume | Total amount | |
|---|---|---|---|---|
| Tube | Dilution factor | ml | isoprene g | conc ng/μl |
| 1 | | 1200 | 0.0816 | 68 |
| 2 | 100 | 590 | | 0.68 |
| 3 | 100 | 590 | | 0.0068 |

For calibration, the following amounts of isoprene were injected into the gas chromatograph (Table 2):

TABLE 2

| Volume injected | | |
|---|---|---|
| μl | Tube | Amount isoprene in ng |
| 0.5 | 3 | 0.0034 |
| 2.5 | 3 | 0.017 |
| 5 | 2 | 3.4 |
| 1 | 1 | 68 |
| 2 | 1 | 136 |
| 5 | 1 | 340 |

A Shimadzu GC-2010 (Shimadzu Deutschland GmbH, Duisburg, Germany) equipped with a flame ionization detector (FID) and a gas chromatography column CP Al2O3, 25 m×0.53 mm 10 μM Film (SGE GmbH, Griesheim, Germany) was used. The analysis was performed isothermally at an oven temperature of 135° C. with a constant flow of the carrier gas nitrogen of 12.2 ml/min and a split ratio of 5. The inlet temperature was 150° C. The detector temperature was 180° C. Samples of 500 μl gas phase were taken by a Shimadzu Combi PAL Headspace Autosampler AOC-5000 automatically injected into the GC. Autosampler setup parameter: syringe temperature: 35° C.; fill speed: 100 μl/s; injection speed: 100 μl/s; syringe flushing: 1 min.

The different amounts of isoprene resulted in peaks of different sizes with different areas under the peak in the chromatogram of the GC. Table 3 shows the correlation between the areas under the peak denoted by “area” in relation to the amount of isoprene:

TABLE 3

| Area | Amount in ng |
|---|---|
| 84 | 0.0034 |
| 424 | 0.017 |
| 6379 | 3.4 |
| 159899 | 68 |
| 287076 | 136 |
| 677149 | 340 |

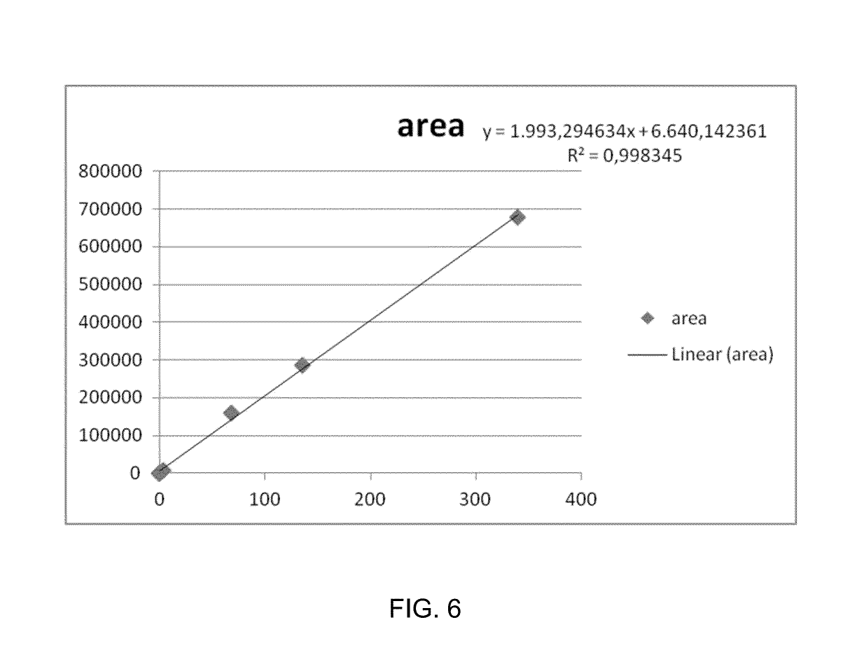

These results were used to calculate the calibration curve shown in FIG. 6, which was then used to determine the amount of isoprene produced by the genetically enhanced cyanobacteria according to the invention.

### Example 4

### Results

### Isoprene Production

First analyses of the created isoprene producing Synechocystis genetically enhanced cells revealed average isoprene production rates (n>5) in the range of 2-62 μg*L⁻¹-*h⁻¹ or 5-270 μg*gDW⁻¹*h⁻¹ depending on the promoter used for IspS expression (see Table 1). The highest production rate was observed for the genetically enhanced cyanobacteria harboring the artificial psaA* promoter controlling transcription of IspS with 117 μg*L-*h⁻¹ or 362 μg*gDW⁻¹*h⁻¹ (duration of 16 hours). This peak value corresponds to 128 μmol isoprene per gram dried cell weight and day at continuous illumination. It is mentionable that the synthetic psaA*-promoter is much more active than other promoters used so far e.g. P_{rbc}, P_{tac/lac} and P_{petJ}, indicating there is still space for further improvement of isoprene production by promoter engineering.

TABLE 4
Average isoprene production rates from at least 5 independent
experiments from different IspS
expressing Synechocystis 6803 strains:

| | | Average | Average |
|---|---|---|---|
| | Average | production | production |
| Genetically | duration | rate in | rate in |
| enhanced strain | [h] | μg * L⁻¹ * h⁻¹ | μg * g_{DW}⁻¹ * h⁻¹ |
| pVZ325-P_{rbcL}-IspSₒₒₚ | 19 | 11.3 [+/− 4.0] | 45.1 [+/− 15.3] |
| pVZ325-lacI-P_{tac}-IspSₒₒₚ | 19 | 2.9 [+/− 0.8] | 12.7 [+/− 6.4] |
| pVZ325-P_{psaA*}-IspSₒₒₚ | 20 | 62.4 [+/− 27.0] | 268.9 [+/− 49.7] |
| pVZ325-P_{petJ}-IspSₒₒₚ | 19 | 1.7 [+/− 0.8] | 5.3 [+/− 3.3] |

From six independent cultivation experiments with the hybrid strain comprising plasmid pVZ325-P_{psaA*}-IspSₒₒₚ (duration between 16-26 hours) an average production rate of about 62 μg isoprene per L culture and hour or 270 μg per gDW and hour was calculated. Compared to the production rates reported by Lindberg et al. (2009) this corresponds to 6.48 mg/gDW*d (about 0.6% of dried cell weight per day). This value is about 130-fold higher than the reported value by Lindberg of 50 μg/gDW*d, for the highest observed value (8.69 mg/gDW*d) even almost 170-fold.

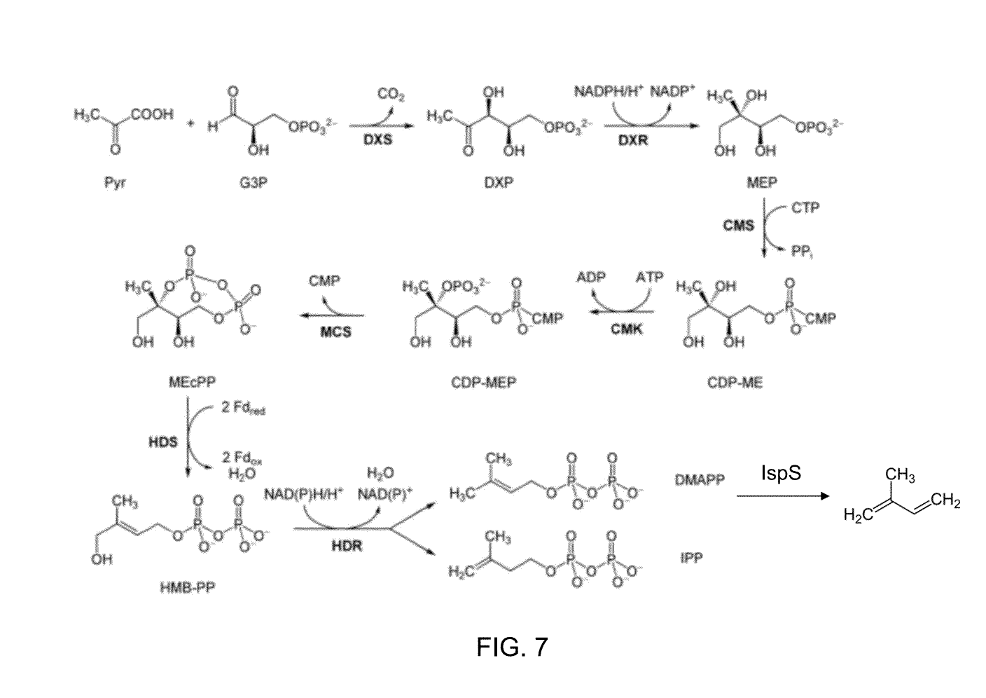

FIG. 7 shows the methylerythritol 4-phosphate pathway leading to isoprene. The abbreviations of the enzymes are:

DXP synthase (DXS) catalyzing the formation of 1-Deoxy-D-xylulose 5-phosphate (DXP), DXP reductase (DXR) catalyzing the formation of 2-C-methylerythritol 4-phosphate (MEP), cytidindiphosphate-Methylerythritol-Synthase (CMS) for the formation of 4-diphosphocytidyl-2-C-methylerythritol (CDP-ME), A cytidyl-methyl-kinase (CMK) for catalyzing the synthesis of 4-diphosphocytidyl-2-C-methyl-D-erythritol 2-phosphate (CDP-MEP), methyl-erythritol-cyclo-diphosphat-synthase (MCS) for the formation of 2-C-methyl-D-erythritol 2,4-cyclopyrophosphate (MEcPP), and hydroxy-methyl-butenyl-diphosphat-synthase (HDS) for synthesizing (E)-4-Hydroxy-3-methyl-but-2-enyl pyrophosphate (HMB-PP). HMB-PP is further converted to dimethylallyl pyrophosphate (DMAPP), which is then converted to isoprene via the isoprene synthase coded by the gene IspS.

The scope of the protection of the invention is not limited to the example given herein above. The invention is embodied in each novel characteristic and each combination of characteristics, which particularly includes every combination of any features which are stated in the claims, even if this feature or this combination of features is not explicitly stated in the claims or in the examples.

We claim:

1. A genetically enhanced cyanobacterium capable of producing isoprene, comprising an extrachromosomal plasmid comprising a gene encoding an enzyme that catalyzes the production of isoprene, wherein said gene encoding the enzyme that catalyzes the production of isoprene is under the transcriptional control of a promoter that comprises a ribosomal binding site and a −10 and −35 region from different cyanobacterial promoters.

2. The genetically enhanced cyanobacterium of claim 1, wherein said extrachromosomal plasmid is an endogenous plasmid.

3. The genetically enhanced cyanobacterium of claim 1, wherein said extrachromosomal plasmid is a heterologous plasmid.

4. The genetically enhanced cyanobacterium of claim 1, wherein the promoter is inducible or constitutive.

5. The genetically enhanced cyanobacterium of claim 4, wherein the promoter is selected from the group consisting of P_{rbc}, P_{tac/lacI}, P_{psaA}, and P_{petJ}.

6. The genetically enhanced cyanobacterium of claim 1, wherein the promoter comprises a ribosomal binding site from an isiA-promoter and a −10 and −35 region from a psaA promoter.

7. The genetically enhanced cyanobacterium of claim 6, wherein the promoter sequence comprises SEQ ID NO: 1,
further wherein the 3′-ATG is the start codon of the gene encoding the enzyme that catalyzes the production of isoprene transcriptionally controlled by this promoter.

8. The genetically enhanced cyanobacterium of claim 4, wherein the promoter comprises a nucleic acid sequence having at least 80% sequence identity to SEQ ID NO: 2, further wherein there is 100% sequence identity at nucleotides 4-9, 26-31, and 45-51 of SEQ ID NO: 2.

9. The genetically enhanced cyanobacterium of claim 1, further comprising a sequence for the termination of transcription that is located downstream of the gene encoding the enzyme that catalyzes the production of isoprene.

10. The genetically enhanced cyanobacterium of claim 1, wherein the gene encoding the enzyme that catalyzes the production of isoprene comprises codon triplets that have been changed in order to enhance translation in the cyanobacterium compared to the respective wild type gene that encodes the enzyme that catalyzes the production of isoprene.

11. The genetically enhanced cyanobacterium of claim 1, wherein the gene encoding the enzyme that catalyzes the production of isoprene is ispS (isoprene synthase—EC 4.2.3.27).

12. The genetically enhanced cyanobacterium of claim 1, wherein the gene encoding the enzyme that catalyzes the production of isoprene has at least 80% sequence identity to the nucleic acid sequence of SEQ ID NO: 8.

13. The genetically enhanced cyanobacterium of claim 1, wherein no further genes involved in isoprene production are present in the cyanobacterium.

14. The genetically enhanced cyanobacterium of claim 1, wherein the cyanobacterium is selected from the group consisting of: Synechocystis, Synechococcus, Anabaena, Chroococcidiopsis, Chlorogloeopsis, Cyanothece, Lyngbya, Phormidium, Nostoc, Spirulina, Arthrospira, Thermosynechococcus BP1, Trichodesmium, Leptolyngbya, Plectonema, Myxosarcina, Pleurocapsa, Oscillatoria, Pseudanabaena, Cyanobacterium, Geitlerinema, Calothrix, Euhalothece, and Scytonema.

15. A genetically enhanced cyanobacterium capable of producing isoprene, comprising an extrachromosomal plasmid comprising a gene encoding an enzyme that catalyzes the production of isoprene wherein said gene is under the transcriptional control of a promoter comprising a sequence of AAGGAGG at its ribosomal binding site, TTGACT at its −10 region, and TATAAT at its −35 region.

16. A genetically enhanced cyanobacterium capable of producing isoprene, comprising an endogenous extrachromosomal plasmid comprising a gene encoding an enzyme that catalyzes the production of isoprene.

17. A method for producing isoprene, comprising the following method steps:
A) culturing the genetically enhanced cyanobacterium of claim 1 in a culture medium, the cyanobacterium thereby producing isoprene; and
B) separating the isoprene from the genetically enhanced cyanobacterium and the culture medium.
